(19) United States
(12) Patent Application Publication
(10) Pub. No.: US 2023/0293055 A1
(43) Pub. Date: Sep. 21, 2023

(54) Smart-Tooth Blood Glucose Measurement Device

(71) Applicant: Joseph John Parsi, San Antonio, TX (US)

(72) Inventor: Joseph John Parsi, San Antonio, TX (US)

(21) Appl. No.: 17/694,751

(22) Filed: Mar. 15, 2022

(51) Int. Cl. A61B 5/145; G01N 21/3563; G01N 21/359; A61B 5/00

(52) U.S. Cl. CPC A61B 5/14532; G01N 21/3563; G01N 21/359; A61B 5/682

(57) ABSTRACT

Disclosed herein is a device and method for a smart-tooth glucose monitoring device. The device is created in response to the desire for an accurate and non-invasive or partially-invasive monitoring device as an alternative to current methods of the glucometer lancet and strips. The device consists of a tooth and its pulp chamber to achieve direct measurement of blood, an optic source and sensors that measure the wavelengths emitted by the optic source. The tooth is embedded with a flexible circuit with a monochromatic light as an optic source with different sensors such as PZT transducer and photodiode within a modified porcelain crown. The monochromatic light pulses passes through the tooth and its pulp chamber and the photoacoustics measured by the sensors which can then be utilized to determine glucose concentration in the blood.

### CROSS-REFERENCE TO RELATED APPLICATIONS

Not applicable

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### Field of the Invention

The present invention generally relates to device and methods for a smart-tooth blood glucose measuring device. More specifically, the present invention generally relates to a device and methods for a smart-tooth blood glucose measuring device for use in non-invasive to partially-invasive measuring and monitoring of blood glucose levels.

### BACKGROUND OF THE INVENTION

Without limiting the scope of the disclosed systems and methods, the background is described in connection with a novel device directed to blood glucose monitoring.

Approximately, 32.4 million or 10.5% of the US population suffer from diabetes. Diabetes is a chronic metabolic disease in which the body loses its ability to control the blood glucose level. This condition over time can lead to multiple organ failure and a compromised life. Continuous control of the glucose level by an individual can stop the progression of the disease, increase compliance and improve the quality of life as well as reduce the mortality and morbidity rate. There are multiple invasive or partially invasive devices to measure a blood glucose level and the ubiquitous of all is the glucometer lancet and strips. All these devices involve the drawing of blood by pricking the skin, which is associated with pain and public stigmatization of dealing with blood and needles.

On the other hand, there has been a never-ending search for a continuous non-invasive or partially invasive device to successfully monitor glucose levels. The least invasive category utilizes optics as the means of measurement. Most optical measurements require light to pass through the skin and the amount of reflection or absorption of the light that can be measured is a factor of glucose content in the blood or through interstitial fluid. To accurately measure the glucose level, all the measurements must occur at one site of the tissue as the tissue architect and its fluid content are different at different parts of the skin. However, the ever-changing epithelial tissue of the skin constantly poses a problem requiring frequent calibration with invasive glucometer pricks and measure. Another issue with the surface skin is the ambient temperature that affects the sensors for measurements as well as the blood flow, and hence the interstitial flood fluctuation. Warmer temperature allows a better flow of fluid under the skin and colder environment restricts flow of fluid in the body. This results in the glucose readings to be different at different temperature.

These major concerns brought upon the idea to have a stable non-changing part of the body to utilize an optical device such as a molar tooth as a site of measuring glucose level. Molar teeth contain a pulp engorged with continuous flow of blood that is surrounded by hard walls of dentin and enamel. In dentistry, the damaged enamel often is shaved off leaving a few millimeters of dentin surrounding the pulp as in a prostatic crown preparation. This closeness to the pulp allows the placement of a NIR or MIR laser light source that easily pass through a few millimeters of dentin. Under this scenario with a few sensors and transducers, it is possible to measure glucose level of the blood in the pulp using photoacoustic spectroscopy.

There are several advantages to this method. One of the advantages is the fact that within the mouth the temperature does not fluctuate as much and remains at body temperature. Furthermore, monochromatic light can easily pass-through dentin of the tooth and pulp chamber, and can therefore be detected by a sensor such as a photodiode. Under this scenario, a crown embedded with a chip with a monochromatic light of a specific wavelength can be utilized to characterize blood for its content and in this case, glucose. Therefore, I used a natural extracted third molar tooth to test this hypothesis.

### BRIEF SUMMARY OF THE INVENTION

Accordingly, it is an object of the present invention to provide a device that allows for noninvasive or partially-invasive glucose monitoring utilizing a tooth.

These and other objects of the present invention are achieved by a device that allows for noninvasive or partially-invasive glucose monitoring utilizing a molar tooth embedded with a chip with a monochromatic light of a specific wavelength.

In an embodiment, the system is comprised of a pulp chamber of a molar tooth, a flexible circuit with an optic source such as a monochromatic light, sensors such as a PZT transducer and photodiode, and a modified porcelain crown that fits the tooth.

Non-invasive or partially-invasive glucose monitoring has been researched extensively on the skin with interstitial fluid beneath. However, this is the first time that it has been shown in vitro, utilizing a tooth and its pulp chamber for direct measurement of blood and not the interstitial fluid. This device displays that pulsed photoacoustic spectroscopy and optic properties can be utilized to determine glucose concentration in the blood. There is a direct correlation between glucose concentration in the blood and the amplitude peak of the registered transducer and photodiode. At the wavelength of 905 nm, it was shown that there is 3.5% increase in peak-to-peak reading of 2.5% Dextrose Water (DW) increase of dextrose in blood and 4.6% increase of peak-to-peak reading of 5% DW increase of dextrose in the blood utilizing photoacoustic spectroscopy (PA).

In summary, the present invention discloses novel device and methods for a smart-tooth blood glucose measuring device. The device and methodology can be used for other components of blood i.e. hematocrit as well as temperature, gyroscope for elder individuals as examples and not a limitation.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a extracted third molar tooth as the site of the blood glucose measurement device in accordance with embodiments of the disclosure;

FIG. 2 is a the molar tooth embed with a flexible circuit with a monochromatic light source with different sensor devices in accordance with embodiments of the disclosure;

FIG. 3 is a top view of the molar tooth embed with a flexible circuit with a monochromatic light source with different sensor devices in accordance with embodiments of the disclosure;

FIG. 4 is a snap shot of data from the oscilloscope in accordance with embodiments of the disclosure;

FIG. 5 is a measurement of bovine blood, bovine blood with 2.5% Dextrose and bovine blood with 5% Dextrose in the tooth pulp chamber absorbed by PZT measured over time in accordance with embodiments of the disclosure;

FIG. 6 is a linear regression plot displaying the linear relationship of PA measurements of bovine blood at zero glucose level baseline, 2.5% Dextrose and 5% Dextrose in accordance with embodiments of the disclosure;

FIG. 7 is a snap shot of data from the oscilloscope in accordance with embodiments of the disclosure;

FIG. 8 is a snap shot of data from the oscilloscope in accordance with embodiments of the disclosure;

FIG. 9 is a snap shot of data from the oscilloscope in accordance with embodiments of the disclosure;

FIG. 10 is a snap shot of data from the oscilloscope in accordance with embodiments of the disclosure;

FIG. 11 is a linear regression plot displaying the linear relationship of PA measurements of bovine blood at zero glucose level baseline, 2.5% Dextrose, 5% Dextrose, 7.5% Dextrose, 10% Dextrose, 12.5% Dextrose in accordance with embodiments of the disclosure;

FIG. 12 is a snap shot of data from the oscilloscope in accordance with embodiments of the disclosure;

FIG. 13 is a linear regression plot displaying the linear relationship of photodiode measurements of bovine blood at zero glucose level baseline, 2.5% Dextrose, 5% Dextrose, 7.5% Dextrose, 10% Dextrose, 12.5% Dextrose in accordance with embodiments of the disclosure.

### DETAILED DESCRIPTION OF THE INVENTION

Disclosed herein is a novel device for a device and methods for a smart-tooth blood glucose measuring device for use in non-invasive or partially-invasive measuring and monitoring of blood glucose levels.

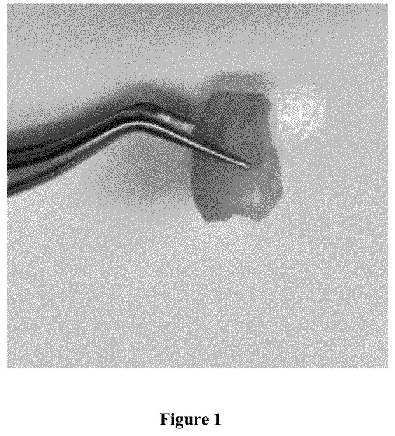

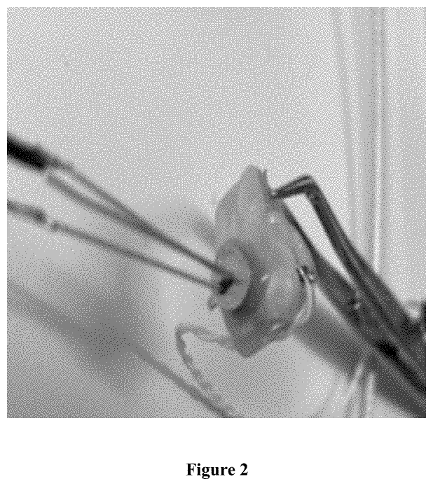

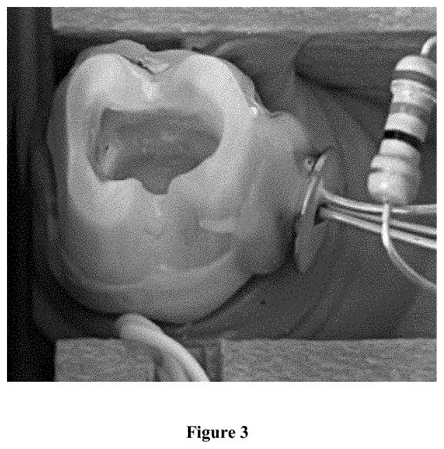

Reference is first made to FIG. 1, FIG. 2, and FIG. 3, a pulp chamber of an extracted molar tooth as the site of blood measurement in accordance with embodiments of the disclosure. The molar is embedded with a flexible circuit with a monochromatic light as an optic source with different sensors such as PZT transducer and photodiode within a modified porcelain crown (see FIGS. 2 and 3). Here, about 3-4 mm of enamel and dentin of the molar tooth was removed on each side (as in porcelain crown preparation) leaving in place 1-2 mm of dentin from the pulp as is displayed on FIG. 3. From the four sides of the molar tooth, one side was used to allow the monochromatic light to pass through and the two other sites were designated for the photodiode and transducer. The upper or crown of the tooth was left intact. Two sources of laser light of 905 nm and 1550 nm (Laser Components) were utilized as these wavelengths have shown to capture the overtone band of glucose [3-4]. The laser light was pulsated at 100 Hz with a pulse generator (Tektronix AFG3051C) and set at the power of 1 watt and pulse width of 1 ns. For the sensor, a photodiode specific for the wavelengths of chromatic light of 800 to 1700 (ThurlabsFDGA05) was selected and the selection for the PZT transducer was a soft A5 level (STEMINC-PIZO) as it has shown to have higher sensitivity in biomedical experiments [5]. In addition, we have used an amplifier (HQA-15M-10T) to boost the weak signal to be registered by an oscilloscope (Tektronix MD03024).

In the chamber of the molar tooth, a different solution containing different concentrations of glucose and water was injected and glucose level was measured and analyzed utilizing the photoacoustic phenomenal in the measurement. Next, we used Bovine Blood (Carolina Biological) mixed with IV 5% DW and 2.5% DW (Hospira 500 ml) in the chamber of molar tooth. The same experiment was repeated and the data was captured from PZT and photodiode using a mixed domain oscilloscope after amplification of the signal.

For the solution preparation, a distilled IV bag of 5% DW was diluted into half each time with saline water to prepare a multiple solution of 5% (original) 2.5, 1.25, 0.75. The monochromatic laser light was set at 1 W, pulse width of 1 ns, and frequency of 100 Hz. PZT transducer and photodiode was connected to oscilloscope and the data was saved for further analysis.

In photoacoustic spectroscopy (PA) NIR electromagnetic wave energy will cause the vibration of molecule at the atomic bonding sites such as in water O—H and in glucose O—H and C—H. This vibration generates thermo-elastic process that upon non-radiating relaxation will generate an acoustic pressure onto the surrounding medium leading to an acoustic sound wave energy that can be detected by placing a transducer in contact with the medium. The intensity of the wave is a reflection of the absorbent of light by the molecules in the solution and its conversion into sound energy. Therefore, if all the variables are kept in a constant state the amplitude of the signal generated from PZT transducer is a reflection of the concentration of the molecule in the solution and in this case concentration of glucose.

First water and glucose with different concentration of 20, 10, 5 percent in the chamber of a molar tooth were used. After passing the monochromatic light of 905 nm through the tooth, the generated signal was captured by PZT transducer and the data was collected by an oscilloscope. The collected data of different concentrations of glucose statistically analyzed and revealed that as glucose concentration increases the amplitude of the signal increases accordingly.

Next, the method is repeated with Bovine Blood and glucose. Due to different cellular component of blood and osmotic effect of distilled water, 5% DW and 2.5% DW mixed with blood was used. Analysis of the data reveals the same results as water and glucose.

In an embodiment the device is comprised of a crown or tooth attachable body configured with a chip (with a power source for the chip) having at least one monochromatic light, at least one PZT transducer, and at least one photo diode. In embodiments, the PZT transducer, as an example and not a limitation, is a crystal or film PZT transducer. In embodiments, the device is configured to have at least one monochromatic light on the opposing side of the tooth of at least one photo diode.

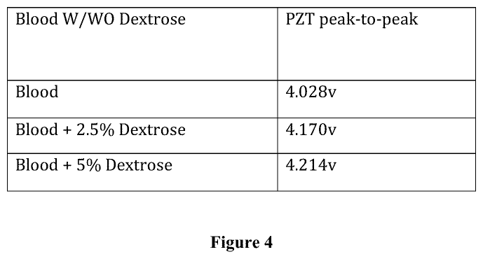

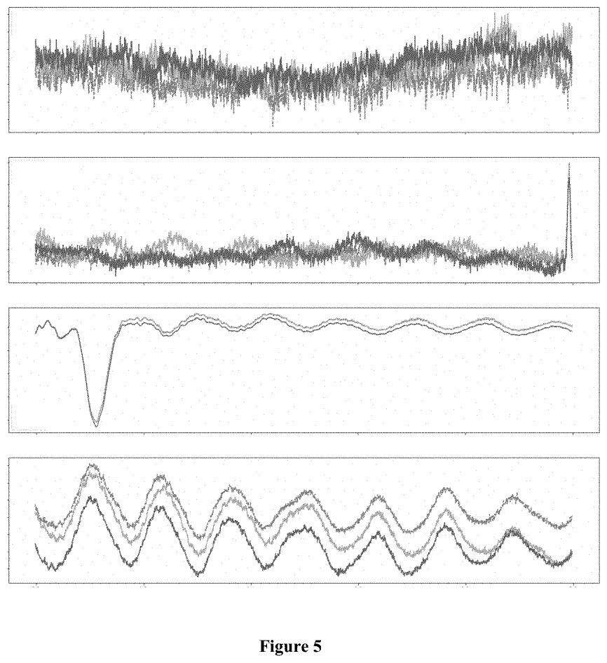

FIG. 4 shows data at a snap shot from oscilloscope. FIG. 5 shows the collective data analysis.

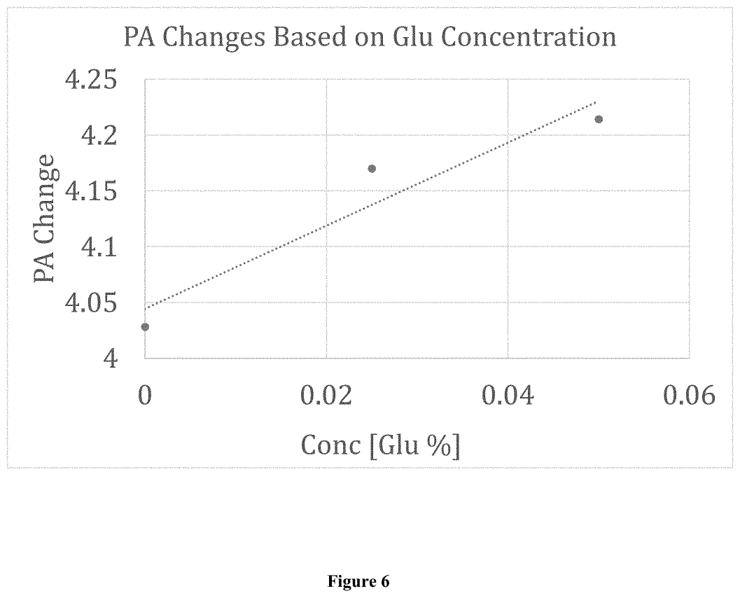

These data revealed that as the concentration of glucose increased the generated signal registered a higher amplitude of the signal by PZT transducer. FIG. 6 is a linear regression model showing the possibilities of estimating glucose concentration and the predicted peak of PZT transducer signal. This reveals the fact that the photoacoustic can be utilized in the tooth to measure glucose level in a continuous monitoring way

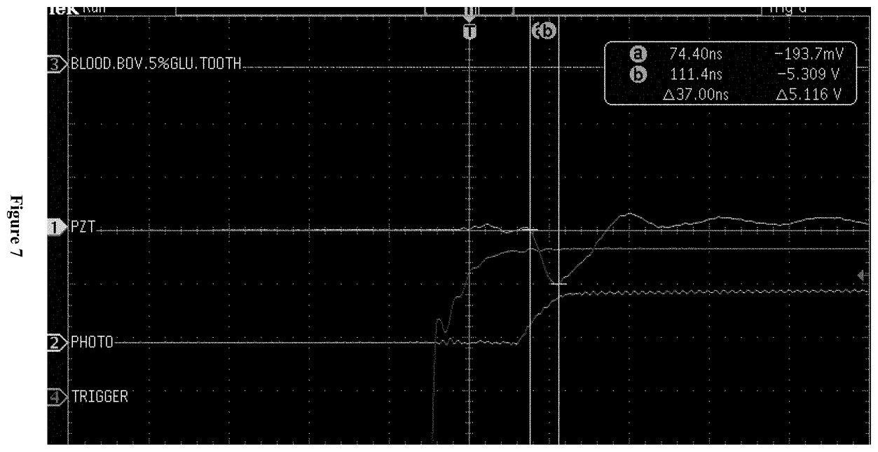

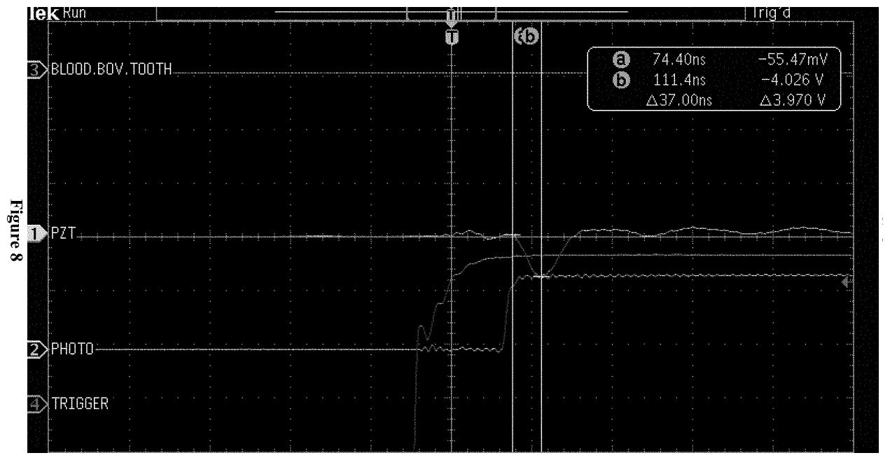

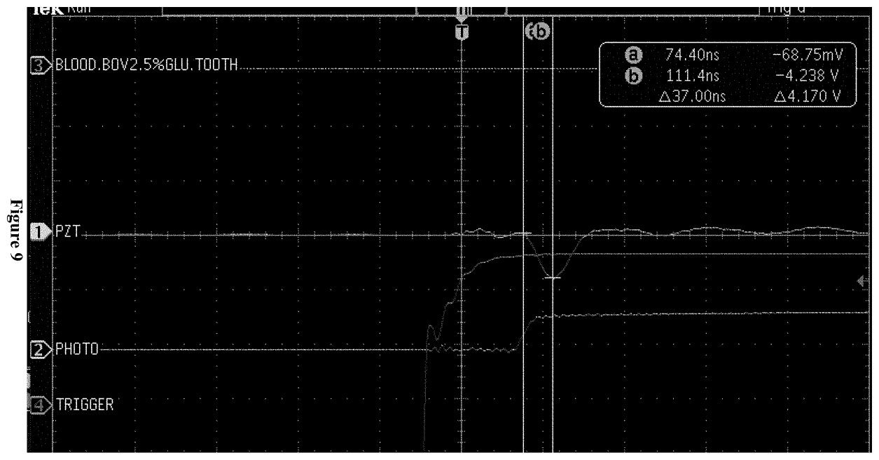

FIGS. 7-9 are some additional results achieved. As we see there is a direct correlation of an increase in glucose concentration and photoacoustic signals or photodiode signals. As the concentration of glucose increases there are more molecules of glucose, and hence increase in acoustic pressure are registered by PZT as its reflected on the oscilloscope. The same correlation exists for glucose concentrations and photodiode: as glucose concentration increases there is less light can pass through the medium and hence the lower level of light reaches photodiode. Therefore; the higher the concentration of glucose the less light and the lesser signal from the photodiode.

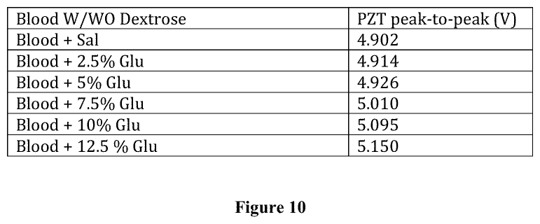

FIG. 10 is a snap-shot of data from the oscilloscope.

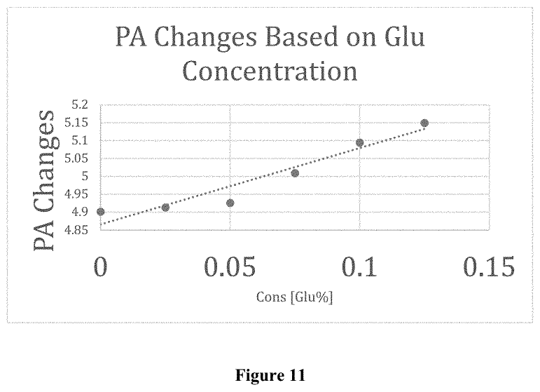

FIG. 11 is a linear regression plot showing the linear relationship of PA measurements of bovine blood at zero glucose level baseline: 2.5% dextrose, 5% dextrose, 7.5% dextrose, 10% dextrose, and 12.5% dextrose.

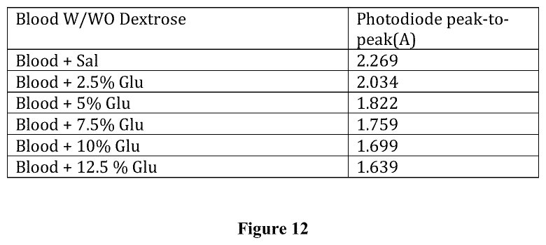

FIG. 12 is a snap-shot of data from the oscilloscope.

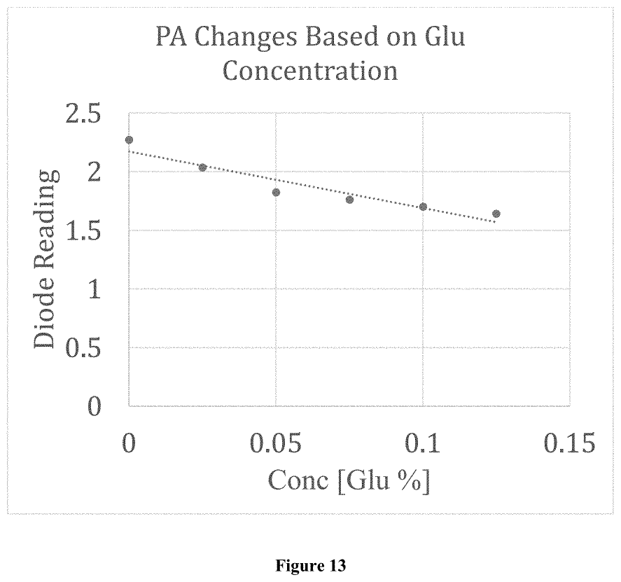

FIG. 13 is a linear regression plot showing the linear relationship of PA measurements of bovine blood at zero glucose level baseline: 2.5% dextrose, 5% dextrose, 7.5% dextrose, 10% dextrose, and 12.5% dextrose.

Although we have set up to measure the photoacoustic effect of the glucose concentration, we also have noticed that photodiode sensor registering lower signal passage as the concentration of glucose in the blood increased. This reflects on the fact that glucose concentration has direct effect on the transmission of light. In other words, as the concentration of glucose increases, glucose molecules absorb more light allowing less light to reach the photodiode.

It is therefore revealed that monochromatic light can pass through the molar tooth and be absorbed by a photodiode. Furthermore, photoacoustic spectroscopy can be conducted in the pulp and different glucose concentration be measured utilizing a transducer such as a PZT. We have shown that the tooth can be used to collect medical necessary data from human body.

In brief, the invention is directed to a smart tooth glucose measuring device. More specifically, the present invention generally relates to a device and methods for a smart-tooth blood glucose measuring device for use in non-invasive or partially-invasive measuring and monitoring blood glucose levels.

The disclosed device and methods is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method of use, except as may be outlined in the claims.

Alternative applications for this invention include using the device or methods in any application where glucose levels are desired.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific devices and methods described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In a further embodiment the device can be utilized so a tooth can be a site for glucose level and other biological determinations such as hematocrit, WBC, body temperature, and gyroscope for falling.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods of use disclosed and claimed herein can be made and executed without undue experimentation in light of the present disclosure. While the systems and methods of this invention have been described in terms of preferred embodiments, it will be apparent to those skilled in the art that variations may be applied to the systems and/or methods in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

### REFERENCES

1. https://www.diabetesresearch.org/file/national-diabetes-statistics-report-2020.pdf
2. A. G. Bell, “Ontheproductionandreproductionofsoundbylight,”Am.J.Sci., vol. 20, 1880, pp. 305-324.
3. A. N. Bashkatov, E. A. Genina, V. I. Kochubey, and V. V. Tuchin, “Optical properties of human skin, subcutaneous and mucous tissues in the wavelength range from 400 to 2000 nm,” Journal of Physics D: Applied Physics, vol. 38, no. 15, pp. 2543-2555, August 2005
4. O. S. Khalil, “Spectroscopic and Clinical Aspects of Noninvasive Glucose Measurements,” Clinical Chemistry, vol. 45, no. 2, pp. 165-177, February 1999
5. Patel C K N & Tam A C (1981) Pulsed optoacoustic spectroscopy of condensed matter. Reviews of Modern Physics 53(3): 517-550

What is claimed is:

1. A tooth glucose measuring and monitoring device comprising of:
a crown or tooth attachable body configured with a chip having at least one monochromatic light, at least one PZT transducer, and at least one photo diode.

2. The device of claim 1, wherein said PZT transducer is crystal transducer.

3. The device of claim 1, wherein said PZT transducer is a film transducer.

4. The device of claim 1, wherein at least one monochromatic light and at least one photo diode are configured to be on opposing sides of a tooth.

5. A method for a tooth blood glucose measuring device as herein disclosed.

6. A tooth measuring and monitoring device for biological determinations such as hematocrit, WBC, body temperature, and gyroscope for falling comprising of:
a crown or tooth attachable body configured with a chip having at least one monochromatic light, at least one PZT transducer, and at least one photo diode.
